(19) United States
(12) Patent Application Publication
(10) Pub. No.: US 2019/0013830 A1
(43) Pub. Date: Jan. 10, 2019

(54) STERILIZABLE WIRELESS COMMUNICATION DEVICES

(71) Applicant: SMART MEDICAL DEVICES, INC., Malibu, CA (US)

(72) Inventors: David H. Hoglund, Malibu, CA (US); Wayne Anderson, Malibu, CA (US); John J. Perry, Malibu, CA (US); Michael M. Karch, Malibu, CA (US); Laura Park, Malibu, CA (US); Edward J. Dohring, Malibu, CA (US)

(21) Appl. No.: 16/030,203

(22) Filed: Jul. 9, 2018

Related U.S. Application Data
(60) Provisional application No. 62/530,677, filed on Jul. 10, 2017.

(51) Int. Cl. H04B 1/3827; H04W 4/80; A61B 17/16; A61B 50/33; A61B 34/35

(52) U.S. Cl. CPC H04B 1/385; H04W 4/80; A61B 2017/00734; A61B 50/33; A61B 34/35; A61B 17/1626

(57) ABSTRACT

Disclosed are devices and methods sterilization wireless communication devices. The devices and methods described involve a sterilizable wireless communication device including a communication module. The communication module includes a transceiver capable of direct wireless communication. The sterilizable wireless communication device further includes a housing having an interior sized to contain the communication module. The communication module is hermetically sealed within the housing and the housing includes a hermetic radio frequency feedthrough configured to couple to an antenna that is external to the housing.

### REFERENCE TO PRIORITY DOCUMENTS

This application claims the benefit of priority to U.S. Provisional Patent Application Ser. No. 62/530,677, filed on Jul. 10, 2017, which is hereby incorporated by reference in its entirety.

### BACKGROUND

Orthopedic surgery can require bone drilling for the repair of fractures or insertion of implants or other devices. The resulting holes can be used to accept screws, implants and other devices to exert pressure, fixation or reduction of the bone or to place prosthetic joints or other implants. Other medical procedures can require access to bone. During any procedure where a drill or other driver is used to advance a tool into and through bone, the user must consciously and carefully limit the penetration to the desired depth. If the user allows the tool to penetrate further, the patient can suffer injury to distal structures such as nerve, brain, spinal cord, artery, vein, muscle, fascia, bone or joint space structures. These types of injuries can lead to severe patient morbidity and even death. The devices inserted to a drilled bore often must fit within a narrow length range that can vary sometimes by no more than a millimeter or less.

### SUMMARY

Aspects of the current subject matter relate to sterilization of wireless communication devices (e.g., autoclavable wireless communication devices).

In one aspect, disclosed is a sterilizable wireless communication device including a communication module. The communication module includes a transceiver capable of direct wireless communication. The sterilizable wireless communication device further includes a housing having an interior sized to contain the communication module. The communication module is hermetically sealed within the housing and the housing includes a hermetic radio frequency feedthrough configured to couple to an antenna that is external to the housing.

In another aspect, disclosed is a sterilizable wireless communication device including a communication module. The communication module includes a transceiver capable of direct wireless communication. The sterilizable wireless communication device further includes one or more input/output connectors configured to directly communicate with an electronic device. The sterilizable wireless communication device further includes a housing having an interior sized to contain the communication module. The communication module is hermetically sealed within the housing.

Also disclosed is a method for forming a forming a sterilizable housing for a wireless communication device. The method includes placing a communication module within a housing, the communication module comprising a transceiver. The housing has a lid and an interior sized to contain the communication module. The method further includes hermetically sealing the communication module within the housing. The housing further includes a hermetic radio frequency feedthrough configured to couple the communication module to an antenna that is external to the housing.

In some variations, one or more features disclosed herein including the following features may optionally be included in any feasible combination. In some aspects, the housing is a microelectronic hermetic housing with a gas tight glass to metal sealing and a ceramic to metal housing. The communication module may be coupled to the hermetic radio frequency feedthrough by a radio frequency cable. The radio frequency cable may include a connector configured to mate with and couple to the hermetic radio frequency feedthrough. The hermetic radio frequency feedthrough may include a sub-miniature push-on, micro (SMPM) connector. The housing may include a stainless steel lid. The stainless steel lid may be laser welded to the housing. The housing may be a microelectronic hermetic housing made of ceramic, metal, and/or other materials. The housing may be configured to be sterilized by autoclave steam sterilization, ethylene oxide sterilization, chlorine dioxide sterilization, hydrogen peroxide sterilization, vaporized hydrogen peroxide sterilization, hydrogen peroxide plasma sterilization, gamma ray sterilization, and/or electron beam sterilization. The communication module may include a Bluetooth Low Energy module. The device may be incorporated inside a body of a medical instrument.

Other features and advantages will be apparent from the following description of various embodiments, which illustrate, by way of example, the principles of the disclosed devices and methods.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. Generally speaking the figures are not to scale in absolute terms or comparatively but are intended to be illustrative. Also, relative placement of features and elements may be modified for the purpose of illustrative clarity.

FIG. 1 is a perspective view of an implementation of an instrument incorporating a sterilizable communications module, in accordance with some example implementations;

FIG. 2 is box diagram schematic showing communication capabilities of the instrument, in accordance with some example implementations;

FIG. 3 is a partial cut-away view of the instrument of FIG. 1 showing a sterilizable wireless communication device incorporated within a body of a durable medical instrument, in accordance with some example implementations;

FIG. 4A is an exploded view of a sterilizable wireless communication device having a housing and a communications module according to a further implementation, in accordance with some example implementations;

FIG. 4B is an exploded view of the housing of FIG. 4A, in accordance with some example implementations;

FIG. 5 is an exploded view of a sterilizable wireless communication device having a housing and communications module according to one implementation;

FIG. 6 is an exploded view of a sterilizable wireless communication device having a housing and communications module according to a further implementation;

FIG. 7A is an exploded view of a sterilizable wireless communication device having a housing and communications module according to a further implementation;

FIG. 7B is an exploded perspective view of a sterilizable wireless communication device having a housing and communications module according to a further implementation;

FIG. 7C is an exploded side view of the sterilizable wireless communication device of FIG. 7B;

FIG. 7D is a side view of the sterilizable wireless communication device of FIGS. 7B and 7C;

FIG. 8A illustrates a top-down view and FIG. 8B is a bottom view of an example implementation of a communications modules;

FIG. 9 is a block diagram and pin out of an example implementation of a communications module;

FIG. 10 is a block diagram of the functional hardware and software for an example implementation of a communications module; and

FIG. 11 is a flowchart illustrating a process for forming a sterilizable wireless communication device, in accordance with some example implementations.

### DETAILED DESCRIPTION

Various forms of surgical equipment sterilization techniques exist including sterilization by autoclave, ethylene oxide, chlorine dioxide, hydrogen peroxide, vaporized hydrogen peroxide, hydrogen peroxide plasma, gamma ray, and electronic beam sterilization. Durable medical instruments that are used, for example, in an operating room need to be re-sterilized before they can be re-used. Autoclave steam sterilization is typically used to sterilize surgical equipment. An autoclave uses elevated temperature, pressure, and steam to sterilize equipment and other objects in order to destroy all bacteria, viruses, fungi, and spores. Autoclave sterilization is best suited for objects capable of withstanding high temperature (+121° C. to +148° C.), high pressure (+1 to +3.5 atm), and high humidity. Durable electronic medical instruments such as orthopedic drills used in surgery have electronics that can be negatively impacted by some types of sterilization techniques. The semiconductors in electronic medical instruments are generally able to tolerate temperatures up to 125° C. However, batteries and wireless communications modules found in durable electronic medical instruments are negatively impacted when exposed to these autoclave conditions.

Some durable electronic medical instruments used in surgery include modular battery packs that can be removed prior to steam sterilization of the medical instrument and then replaced using sterile technique prior to re-use. Some medical instruments protect certain electronic components with plastic or silicone potting materials such that they need not be removed from the medical instrument and can undergo sterilization. Potting involves filling a complete electronic assembly with a solid or gelatinous compound such as thermosetting plastics or silicone rubber gels to resist shock, vibration, moisture and/or corrosive agents from infiltrating the electronics. Despite these advances, durable electronic medical instruments that require re-sterilization between uses have been limited and generally do not include sensitive electronics for wireless communications because they are unable to withstand autoclave or steam sterilization conditions.

The present disclosure relates generally to durable medical instruments having electronics for wireless communications that need not be removed from the instrument such that the entire medical instrument can undergo sterilization, in particular, repeat steam sterilization such as by autoclaving, before re-use. While many of the examples relate to durable medical instruments, the sterilizable communication devices described herein may be incorporated into other devices as well.

The sterilizable electronics for wireless communications may be incorporated into a medical instrument, such as a cordless orthopedic bone drill system or other medical instrument, capable of wireless communications to an operating room computer or heads-up display. For example, the medical instrument can sense data (e.g. detect spindle torque, feed force, and feed rate) in real-time and transmit that data wirelessly to computing technology in the operating room. Data that is transmitted can be displayed numerically and/or graphically for the operator during a procedure. As will be described in more detail below, these systems and others can include electronics and/or communications modules housed in a manner configured to withstand thousands of cycles of autoclave steam sterilization. For example, the instruments described herein incorporate one or more housings and electronic feeds that are hermetically sealed to reliably encapsulate electronic and/or wireless components with gas-tight Glass to Metal Sealing, Ceramic to Metal, and/or Full Ceramic housings. The housings ensure the wireless communication modules of the instruments are undamaged even after thousands of autoclave or sterilization cycles.

The sterilizable electronics for wireless communications may also be incorporated into a surgical tray or other equipment for tracking purposes. For example, a surgical tray may hold medical instruments that include an identifier and/or a wireless communications system, such as a radio frequency identification (RFID) tag, a GPS location module, a communication antenna, and/or the like. The surgical trays, along with the medical equipment, may be subjected to sterilization and it may be desirable to track the location and/or sterilization status of the surgical trays and/or the medical equipment disposed on the surgical trays.

Instruments

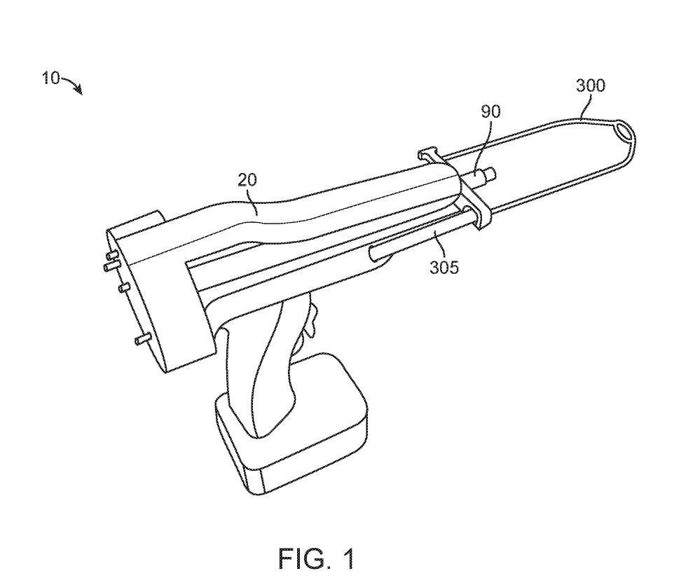

Turning now to FIG. 1, the instrument 10 can include a body 20 that houses a power system configured to move a working tool. The working tool can be a drill bit, saw, burr, reamer, Kirschner (or other) wire, pin, trochar, screw driver, wrench, router, router bit, stepped drill bit, bone plug removal tool, bone harvesting tool, bone marrow harvesting tool, bone marrow aspirating tool, self-drilling screw, or other tool, cutting element, or driving element. The power system can be one or more of motor, rotational drive motor, pneumatic motor or actuator powered by a gas source, electrical motor, hydraulic actuator, and the like.

In some implementations, the instrument 10 can instantaneously sense, meter and control the work created by the working tool. For example, the torque, power usage and/or the energy can be sensed, metered, and reported to the operator graphically and/or numerically and/or with gauges. Instantaneous sensing, metering and controlling the instrument 10 can help to prevent injury to surrounding tissues and structures that could otherwise be caused by the working tool. For example, sensing, metering and controlling the rotational speed of the drive can reduce the risk of heating surrounding tissue and bone, for example to the point of causing localized burns. Sensing, metering and controlling the axial motion and/or relative extension of the working tool can prevent penetrating injuries, for example, to structures distal of the target such as nerve, brain, spinal cord, artery, vein, muscle, fascia, bone or joint space structures. The instrument 10 can include any of the implementations described in U.S. Pat. Nos. 8,821,493; 9,526,511; 8,894,654; and International Patent Application No. PCT/US2017/017517, filed Feb. 10, 2017.

Still with respect to FIG. 1, the instrument 10 can include one or more guides such as a guide harp 300 configured to be withdrawn in a proximal direction to reveal a length of the working tool extending beyond the distal engagement end of the instrument 10. The guide harp 300 can include two or more supporting arms or rods 305 positioned symmetrically around the central, longitudinal axis A of the working tool. The symmetrical orientation of the guide harp 300 around the central longitudinal axis A that is coaxial with the direction of force applied by the working tool prevents the guide from acting like a lever arm. It should be appreciated that the harp 300 can be designed to incorporate one arm. In this implementation, a distal part of the arm can bend towards and surround the working tool, which would allow the working tool to act as a functional support arm to stabilize the construct from levering or moving off of the longitudinal axis. The axis of the guide harp 300 is aligned with the axis of the working tool which is aligned with the direction of axial force being applied to increase stability of the instrument 10 and avoids the guide harp 300 from inadvertently causing pivoting movements away from the z-axis. The guide harp 300 can have one, two, three, or more rods 305 that provide support to bear the load. The rods 305 of the guide harp 300 can be singular units or can have telescoping rods. Telescoping rods can provide the instrument 10 with a larger range in overall penetration length in a more efficient configuration and eliminate the rods 305 from exiting the back end of the drill. The telescoping rods can each include an actuator such as a pneumatic, hydraulic, motorized or other actuator that causes the guide harp 300 to telescope and change overall guide length (i.e. telescope outward to lengthen or telescope inward to shorten).

The instrument 10 can incorporate actuators such as one or more triggers, buttons, and switches that can be retracted, pressed, squeezed, slid or otherwise actuated to perform a certain function of the instrument 10. The actuators can be incorporated into a handle of the instrument 10 in such a way that is ergonomically comfortable for a user. For example, the instrument can include a pistol grip handle having trigger-type actuators such that the instrument 10 can be easily and comfortably held and actuated during use. The pistol grip handle can include a lip under the actuators for the fingers to press against. It should be appreciated, however, that the instrument 10 can have other configurations such as a straight-bodied instrument that does not include a pistol grip handle. Although the above describes the use of “triggers” or “actuators” to cause a particular action of the instrument 10, it should be appreciated that triggers and actuators can include foot pedals to cause a particular action in the instrument. The instrument 10 may also be actuated or triggered by programming the instrument 10 to perform a particular action via a user interface on the instrument 10 or using an external computing device remote from the instrument 10 that is in wired or wireless communication with the instrument, which will be described in more detail below.

Power and Electronics

The instrument 10 can be a cordless powered instrument. In an implementation, the instrument 10 includes and is powered by a removable battery pack. The battery pack can be enclosed within a battery cover capped on the bottom by a battery case cover that can be removed, for example, upon depression of a battery release button. The circuit board for the electronics can be sandwiched above the battery such that the electronics all drop out upon removal of the battery. The battery can have different chemical compositions or characteristics. For instance, batteries can include lead-acid, nickel cadmium, nickel metal hydride, silver-oxide, mercury oxide, lithium ion, lithium ion polymer, or other lithium chemistries. The instruments can also include rechargeable batteries using either a DC power-port, induction, solar cells or the like for recharging. Power systems known in the art for powering medical devices for use in the operating room are to be considered herein. It should be appreciated that other power systems known outside the art of medical devices are to be considered herein as well.

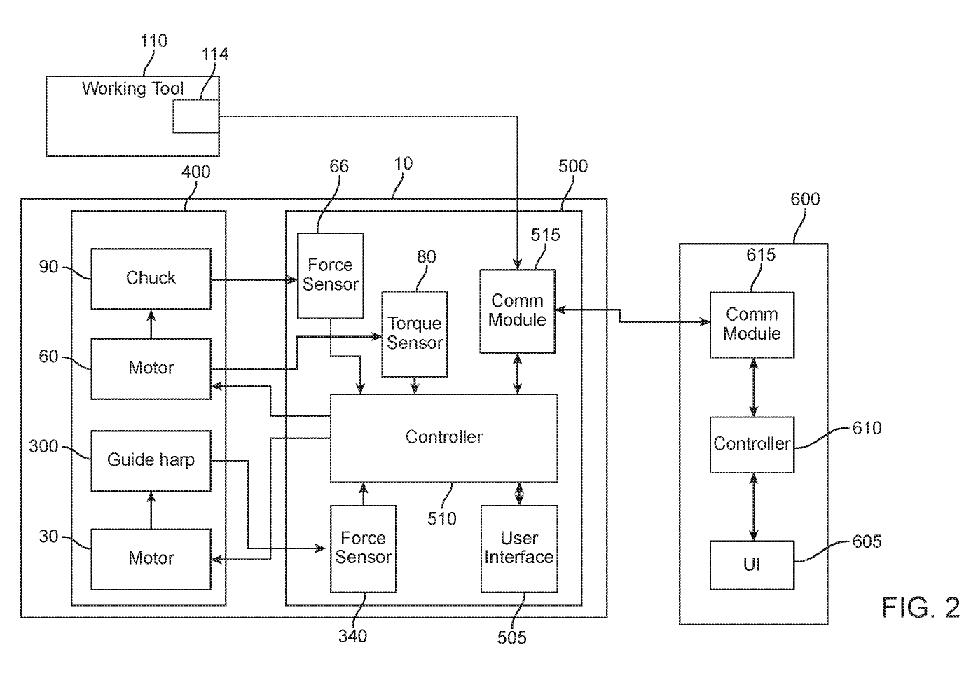

FIG. 2 is a block diagram illustrating an implementation of the instrument 10 having a drive module 400 in communication with an electronics module 500. The drive module 400 can include a working tool 110 and configured to be driven by a motor 60. The electronics module 500 of the instrument 10 can include a user interface 505, a controller 510, communication module 515, and the one or more sensors of the instrument 10 including, but not limited to force sensors 66, 340 and/or torque sensors 80. The controller 510 may be in operative communication with one or more components of the drive module 400 as well as in operative communication with one or more components of the electronic module 500 including the sensors, communication module 515 and user interface 505. The various sensors can communicate information in real-time to the controller 510 such that it can be displayed to the user via the user interface 505 on the instrument 10.

The user interface 505 can receive manual input from a user and may include at least one actuator, trigger, pushbutton, keypad, touchscreen, or other input. The user interface 505 may include at least one light, screen, display or other visual indicator to provide instructions and/or information to the user, such as when to stop drilling. The user interface 505 may include auditory or tactile indicators as well. For example, the user interface 505 can provide the user with alerts and information regarding the status of the instrument 10 and instrument components during use such that manual and/or automatic adjustments can be made. The user interface 505 can include an LED or other type of display using, for example, electrical filaments, plasma, gas or the like. The user interface 505 can include a touch-screen type of display. It should be appreciated that the instrument 10 need not include a user interface 505 and instead communicate with an external computing device 600 having a user interface 605.

The controller 510 can include at least one processor and a memory device. The memory may be configured for receiving and storing user input data as well as data acquired during use of the instrument 10 such as from the one or more sensors. The memory can be any type of memory capable of storing data and communicating that data to one or more other components of the device, such as the processor. The memory may be one or more of a Flash memory, SRAM, ROM, DRAM, RAM, EPROM, dynamic storage, and the like. The memory can be configured to store one or more user-defined profiles relating to the intended use of the instrument 10. The memory can be configured to store user information, history of use, measurements made, and the like.

The communication module 515 is configured to communicate with another device. In some implementations, the communication module 515 can communicate with the working tool 110 as will be described in more detail below. In some implementations, the communication module 515 can communicate with an external computing device 600. The external computing device 600 can incorporate a communication module 615, a controller 610 and a user interface 605 (such as a graphical user interface or GUI). The communication module 515 of the instrument 10 and also the communication module 615 of the external computing device 600 can include a wired communication port such as a RS22 connection, USB connection, Firewire connections, proprietary connections, or any other suitable type of hard-wired connection configured to receive and/or send information to the external computing device 600. The communication module 515 and also the communication module 615 of the external computing device 600 can alternatively or additionally include a wireless communication port such that information can be fed between the instrument 10 and the external computing device 600 via a wireless link, for example to display information in real-time on the external computing device 600. The wireless connection can use any suitable wireless system, such as Bluetooth, Wi-Fi, radio frequency, ZigBee communication protocols, infrared or cellular phone systems, and can also employ coding or authentication to verify the origin of the information received. The wireless connection can also be any of a variety of proprietary wireless connection protocols. As mentioned above, in some implementations, the instrument 10 has no user interface 505 and communicates with the external computing device 600 configured to display information related to the instrument 10. The external computing device 600 can also control the instrument 10 such that the communication between the instrument 10 and the external computing device 600 is two-way communication.

The external computing device 600 with which the instrument 10 communicates can vary including, but not limited to, desktop computer, laptop computer, tablet computer, smartphone or other device capable of displaying information and receiving user input. The user interface 605 of the external computing device 600 can display information regarding the use of the instrument 10 relayed in real-time and provided to a user instantaneously during use of the instrument 10. The information can vary, including for example, bore depth, energy, power, torque, force, time or other information as will be described in more detail below. The user interface 605 of the external computing device 600 can also include one or more inputs such as a touchscreen or other inputs including buttons, keys, touchpads, or the like such that a user can interact with the processor to perform certain actions related to the programming of the instrument 10. The user interface 605 of the external computing device 600 can include a touchscreen. The controller 610 of the external computing device 600 can include at least one processor and a memory device as described in more detail above with respect to controller 510.

The external computing device 600 can be a heads-up display that communicates with the instrument 10 (i.e. either wired or wirelessly) and having a graphical user interface (GUI) that can display data and provide interactive functions such as a touch screen for input of data and information about the instrument 10. The heads-up display can be mounted as is known in the art such as with a boom or other mechanism that provides user convenience. For example, the heads-up display can be mounted on a boom that can be easily positioned and moved around during a surgical procedure. The heads-up display can be autoclavable such that the display can be positioned within the surgical field where a user is using the instrument 10. Alternatively, the heads-up display can be inserted into a sterile cover such that the display can be positioned within the surgical field where a user is using the instrument 10.

As mentioned, the communication module 515 can communicate with the working tool 110. In some implementations, the communication module 515 can communicate with a transponder or other data element 114 on the working tool 110 configured to be in communication with the communication module 515. As an example, the element 114 can store data about the working tool 110 such as diameter, length, number of previous uses, date of manufacture, as well as any other information regarding the working tool 110. The data can be stored within the element 114 and communicated to and received by the controller 510 of the instrument 10 upon “reading” the element 114 on the working tool 110. The identification of the working tool 110 can be used by the controller 510 to set or to adjust certain parameters. The data can be received as part of a set-up procedure and preparation of the instrument for actual use. This can be initiated automatically by software run by the controller 510 of the instrument 10 without any user input. For example, diameter of the working tool 110 can be important in providing information regarding bone density and length of the working tool can be important for zeroing the instrument prior to drilling. The communication can be one-way or two-way wireless communication. The communication can be a wireless communication such as a transmitter and/or receiver, radiofrequency (RF) transceiver, WIFI connection, infrared or Bluetooth communication device. The data element 114 of the working tool 110 can include an encoder or bar code type strip configured to be scanned and read by a corresponding reader device of the instrument 10 that is in operative communication with the controller 510. The data element 114 may alternatively be an RFID chip or the like that transmits data to a reader such as a data receiving processor or the like. Such encoder devices include the ability to securely transmit and store data, such as, via, encryption, to prevent unauthorized access or tampering with such data.

The memory of the controller 510 can be configured to maintain a record for a particular working tool 110. For example, the record can indicate when the tool 110 is sufficiently dull that it should not be used for a particular operation. Once a tool 110 has reached a particular threshold for dullness, such as data regarding total energy of the tool, the software can be configured to write onto the memory of the data element 114 of the working tool 110 such that upon subsequent use, the instrument 10 is alerted to the information that the working tool 110 should not be used. Thus, information can be sent between the instrument 10 and the working tool 110 in a two-way manner.

Hermetic Housings

Durable medical instruments that are used, for example, in an operating room need to be re-sterilized before they can be re-used. One or more of the electronic components of the instruments described herein can be reversibly removed from the instrument. For example, the body 20 can include one or more removable covers that can be used to access one or more of the various internal components. Further, one or more of the internal components can be modular and can be completely separated from the body 20 of the instrument 10. This allows for interchanging parts as well as cleaning and sterilizing the components of the instrument 10. For example, the battery pack can be removable from the instrument 10, for example, during autoclaving. Similarly, one or more components of the electronics module 500 and/or the drive module 400 can be modularly removable for easier cleaning and autoclaving.

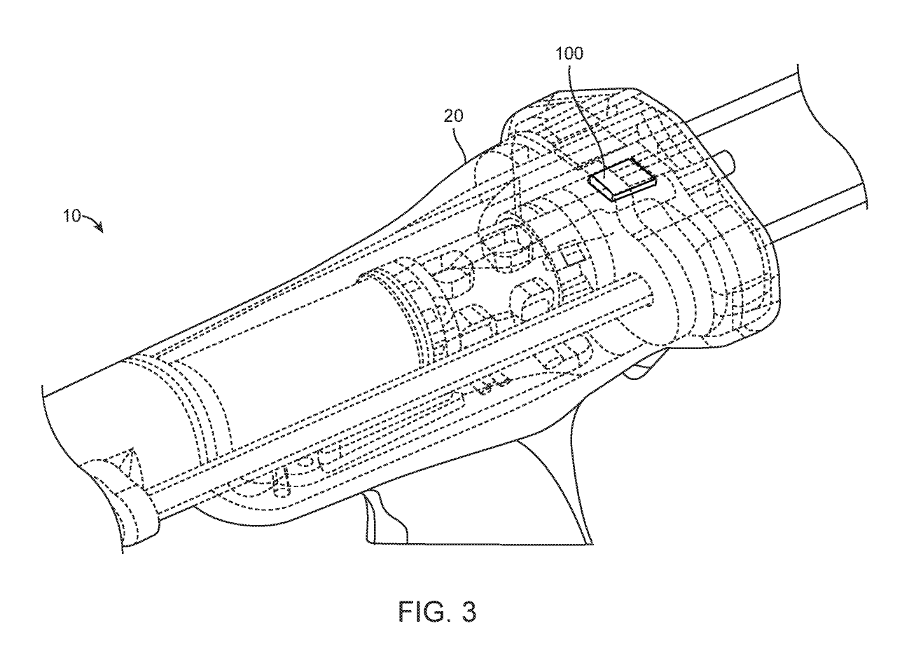

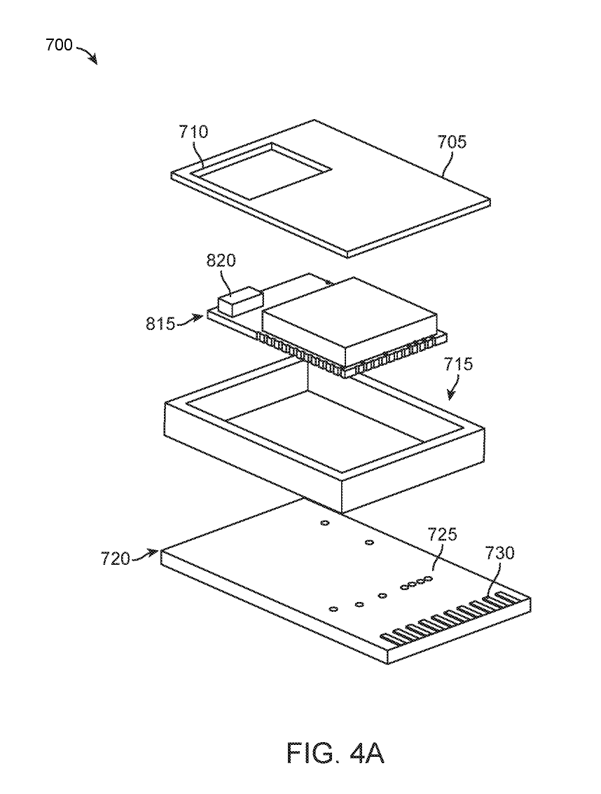

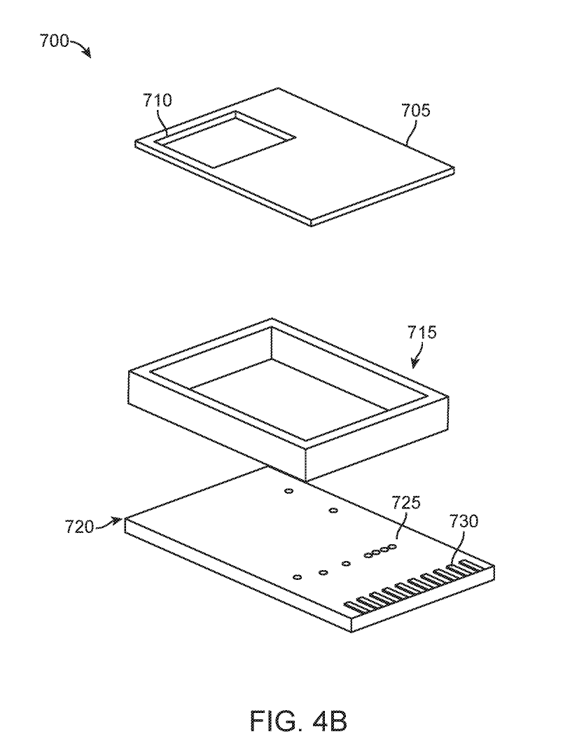

Some components of the electronics module 500 need not be removed from the instrument 10 prior to sterilization. FIG. 3 illustrates an instrument 10 incorporating a sterilizable wireless communication device 100 located within the body 20 of the instrument 10. As best shown in FIGS. 4A-4B, the sterilizable wireless communication device 100 can include one or more components of the communication module 815 hermetically sealed within a housing 700. The housing 700 is able to withstand repeated autoclave (steam sterilization) cycles, at least about 3,500 cycles or more.

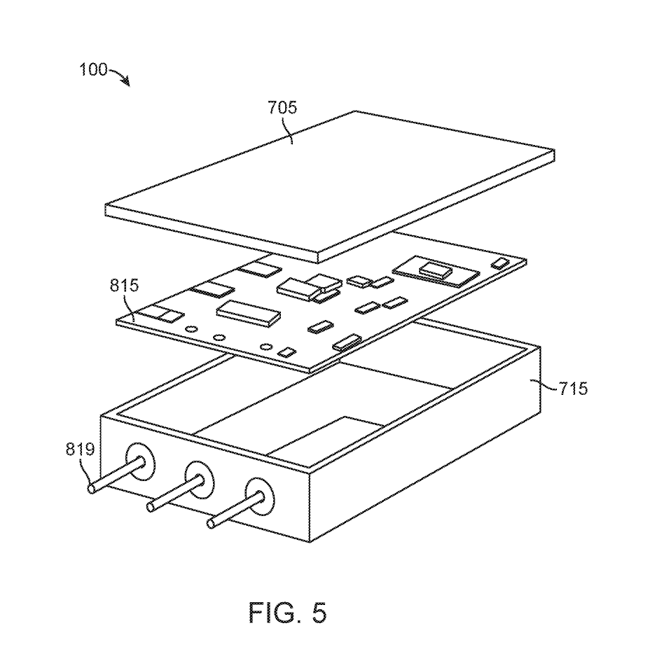

The housing 700 can be a microelectronic hermitic housing with gas-tight Glass to Metal Sealing (GTMS) (see FIG. 5). The housing 700 containing the communications module 815 can be sealed by creating an airtight hermetic seal between glass and a metal package. This glass-to-metal sealing involves running an isolated electrical current through a metal wire from outside the metal package to the inside. Molten glass wets the metal in order to form a tight bond and thermal expansion of the glass and metal closely match to maintain a solid seal as the assembly cools. In some implementations, the inner material has a coefficient of expansion that is slightly less than the outer material such that the seal tightens as it cools.

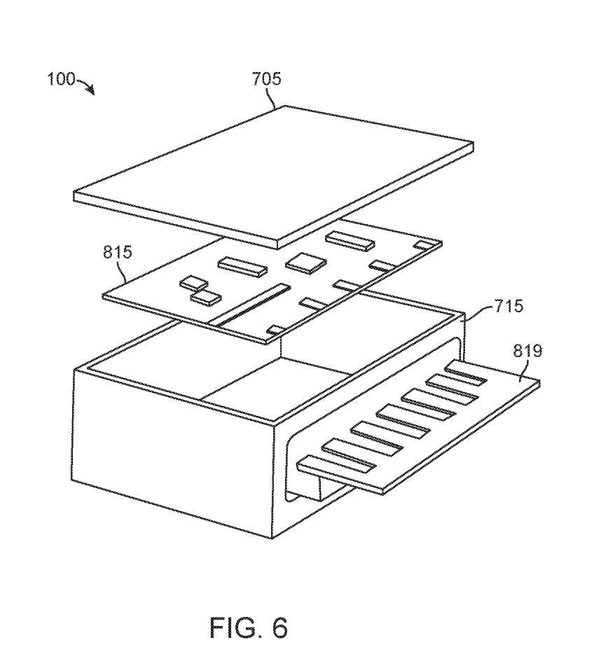

The housing 700 can be a microelectronic hermitic housing with Ceramic to Metal (CeRTMS) (see FIG. 6). In the formation of a glass-ceramic-to-metal seal, the parts to be joined are first heated, normally under inert atmosphere, in order to melt the glass and allow it to wet and flow into the metal parts. The temperature can then be reduced into a temperature regime where many microscopic nuclei are formed in the glass. The temperature is then raised again into a regime where the major crystalline phases can form and grow to create the polycrystalline ceramic material with thermal expansion characteristics matched to that of the particular metal parts.

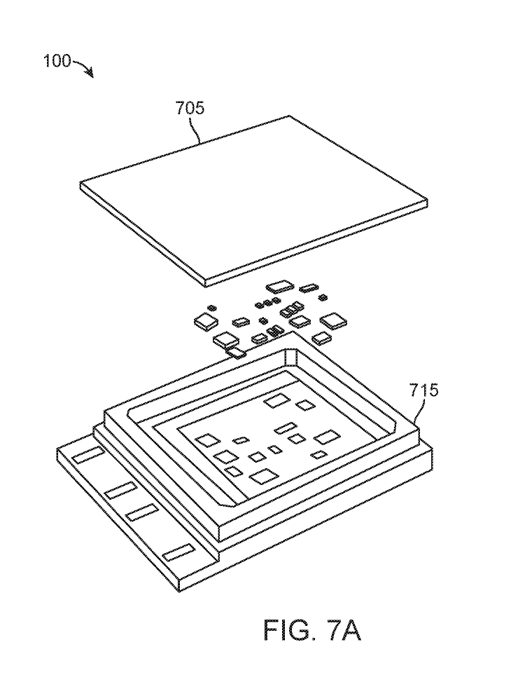

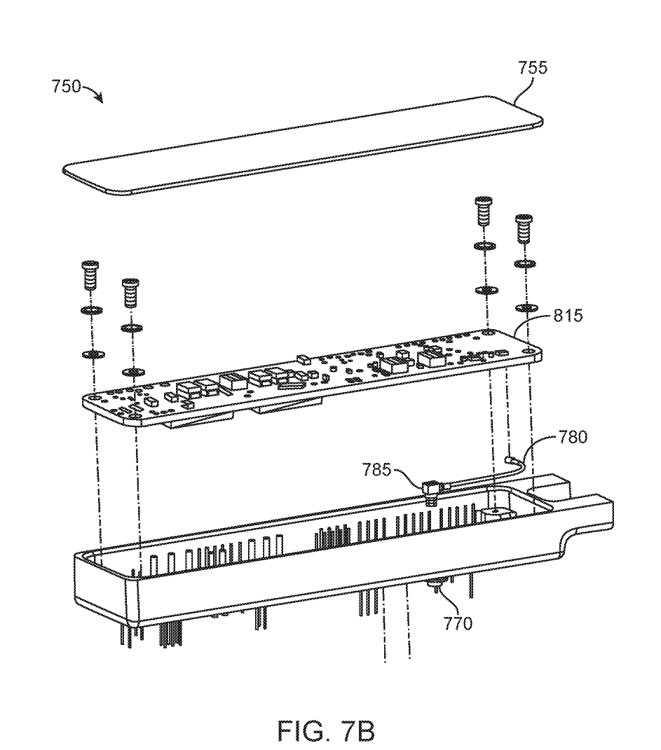

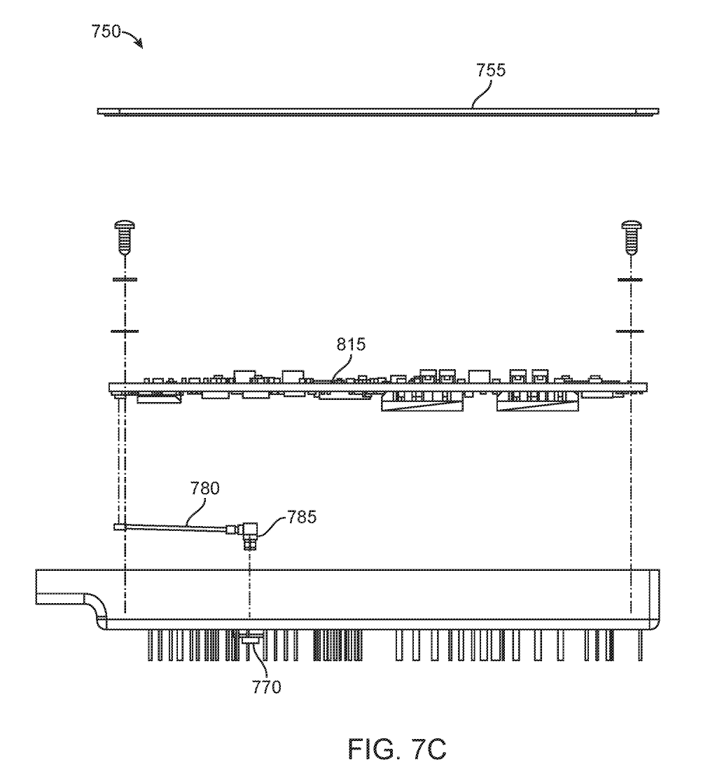

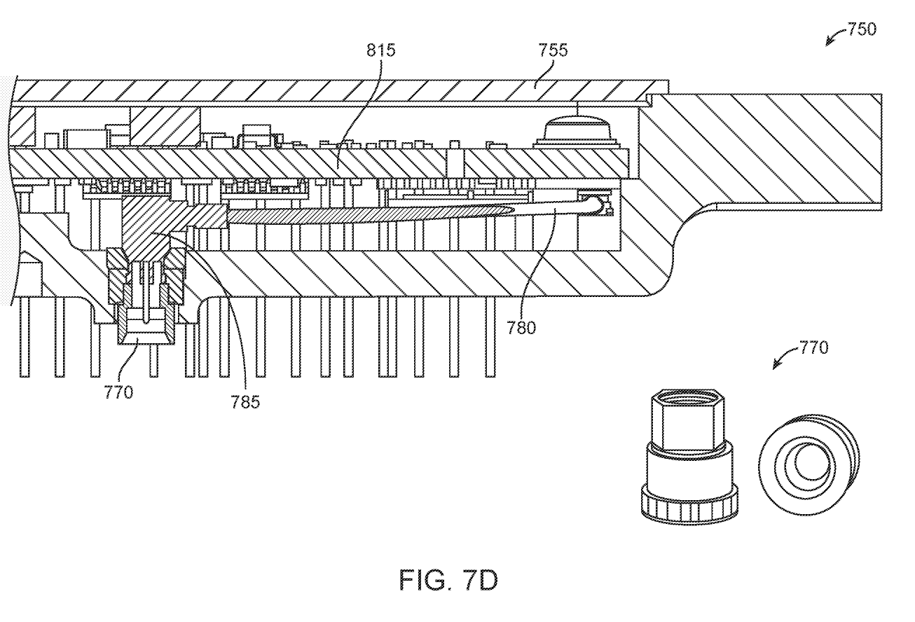

The housing 700 can be a microelectronic hermitic housing with full ceramic packaged housing (see FIG. 7A), which is laser-welded. A ceramic is an inorganic, non-metallic, solid material that includes metal, non-metal or metalloid atoms primarily held in ionic and covalent bonds. Laser-beam welding (LBW) is a welding technique used to join multiple pieces of metal (and can be used to weld ceramics together) through the use of a laser. The beam provides a concentrated heat source, allowing for narrow, deep welds and high welding rates. The process is frequently used in high volume applications using automation, such as in the automotive industry. It is based on keyhole or penetration mode welding. Like electron beam welding (EBW), LBW has high power density (on the order of 1 MW/cm²) resulting in small heat-affected zones and high heating and cooling rates. The spot size of the laser can vary between 0.2 mm and 13 mm, though smaller sizes are used for welding. The depth of penetration is proportional to the amount of power supplied, but is also dependent on the location of the focal point: penetration is maximized when the focal point is slightly below the surface of the workpiece.

The communication module 815 can include a transmitter, receiver, and/or transceiver having an antenna 820 capable of direct wireless communication with one or more wireless communication devices. The transceiver can be a Bluetooth communication device, such as Bluetooth Low Energy or Bluetooth Smart. The transceiver may also include any radiofrequency (RF) transceiver for wireless communication, such as a transceiver for Wi-Fi, cellular, infrared, near-field communication (NFC), Zigbee, ultra-wideband, and/or the like. One or more I/O connectors 819 can be incorporated that allow direct communication with other electronic devices.

FIGS. 4A-4B illustrate exploded views of a housing 700 enclosing by a hermetic seal a communications module 815 having an antenna 820. The housing 700 can include a lid 705, a frame 715 and a substrate 720 having pin locations 725 and a connector array 730. The lid 705 can be formed of cobalt. Because metals and ceramics can block radio signals, the lid 705 can include a window 710 fused to it that allows transmission of radio waves from the antenna 820 to the outside of the hermetic seal of the housing 700. The window 710 can be formed of sapphire, quartz, cobalt, or any material that allows transmission and reception of radio waves from the antenna within the interior of the housing to outside of the housing. In the example of FIGS. 4A and 4B, the sapphire window 710 is positioned over the antenna 820 allows for broadcast and reception of signals. The antenna 820 can also be external to the hermetically sealed chamber and connect to the transceiver through input/outputs (I/O). The substrate 720 can be an HTCC ceramic substrate (e.g. 1 mm thick). The housing 700 can be gas-tight to 1×10 mbar×1/s and have temperature stability greater than 250° C. and a thermal shock stability to −65° C. to 150° C. The housing 700 can include electric insulation to greater than 10 G Ohms. The housing 700 can be steam sterilized up to about 2 bar and 134° C. and may withstand autoclave cycles of at least 3,500 cycles.

In some implementations, the frame 715 can be coupled to the substrate 720 such that bottom edges of the walls of the frame 715 sit atop an upper surface of the substrate 720 surrounding the pin locations 725 such that they remain internal to the frame 715 and the connector array 730 external to the frame 715. A lower surface of the lid 705 can couple to upper edges of the walls of the frame 715 thereby forming an enclosed interior to the housing 700 formed by the upper surface of the substrate 720 internal surfaces of the walls and the lower surface of the lid 705. The communications module 815 and antenna 820 can be enclosed within this internal volume such that the one or more pins of the communication module 815 can couple with the pin locations 725. In other implementations, the I/O connectors 819 extend through a wall of the frame 715 (see FIGS. 5 and 6). The I/O connectors 819 may include ceramic, dielectric glass, and/or another suitable material. The instrument 10 can also incorporate an autoclavable multi-pin connector configured to undergo steam sterilization. The connector can include a plurality of small glass-to-metal sealed signal lines and two power pins for feedthrough.

FIGS. 7B-7C illustrate exploded views of a housing 750 enclosing by a hermetic seal a communications module 815. The housing 750 can include a lid 755, a frame 765 having a radio frequency (RF) feedthrough 770. The lid 755 may be formed of stainless steel, cobalt, ceramic, and/or the like. The frame 765 may be formed of stainless steel, cobalt, ceramic, and/or the like. The communications module 815 may couple with an RF cable 780 that connects to the RF feedthrough 770. The RF cable may include a connector 785 configured to mate with and couple to the RF feedthrough 770. The RF feedthrough 770 may be configured to connect the communications module 815 to an antenna 815 external to the housing 750. In some aspects, the RF feedthrough 770 is a sub-miniature push-on, micro (SMPM) connector or any other RF connector configured to connect the hermetically sealed communications module 815 to an external antenna 820. The housing 750 may be sealed with a GTMS, a CeRTMS, a laser-weld, and/or the like. FIG. 7D illustrates a side view of the housing 750 of FIGS. 7B and 7C.

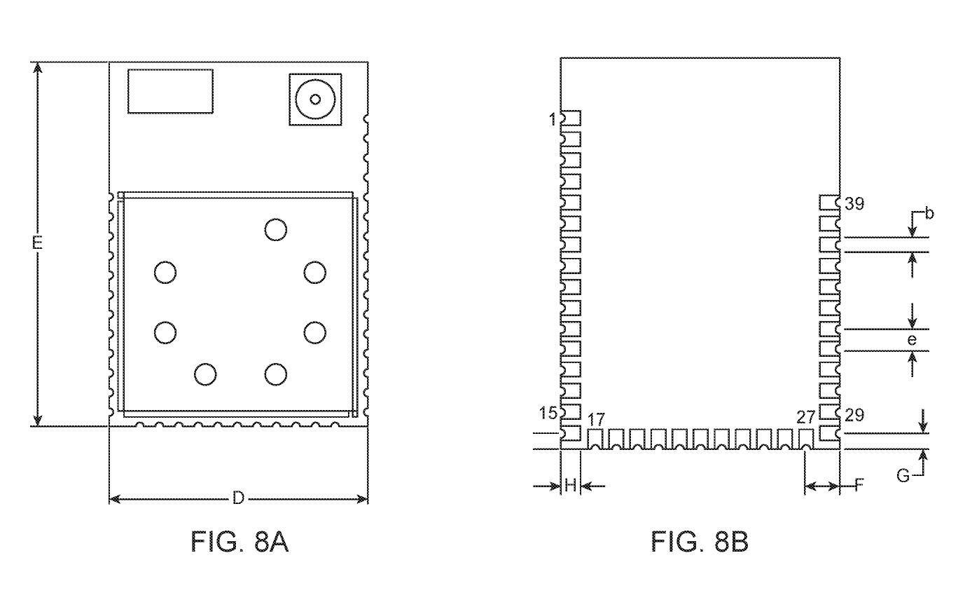

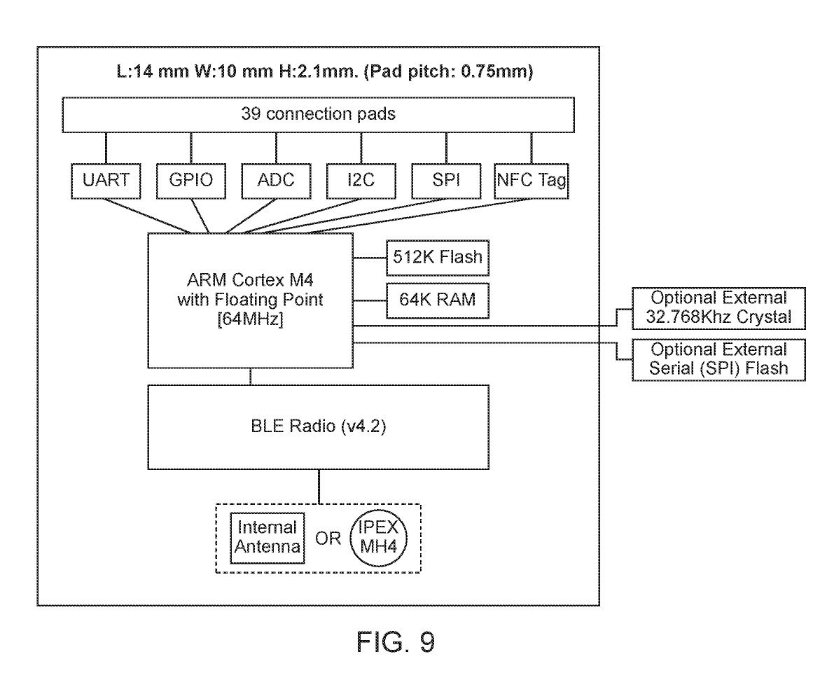

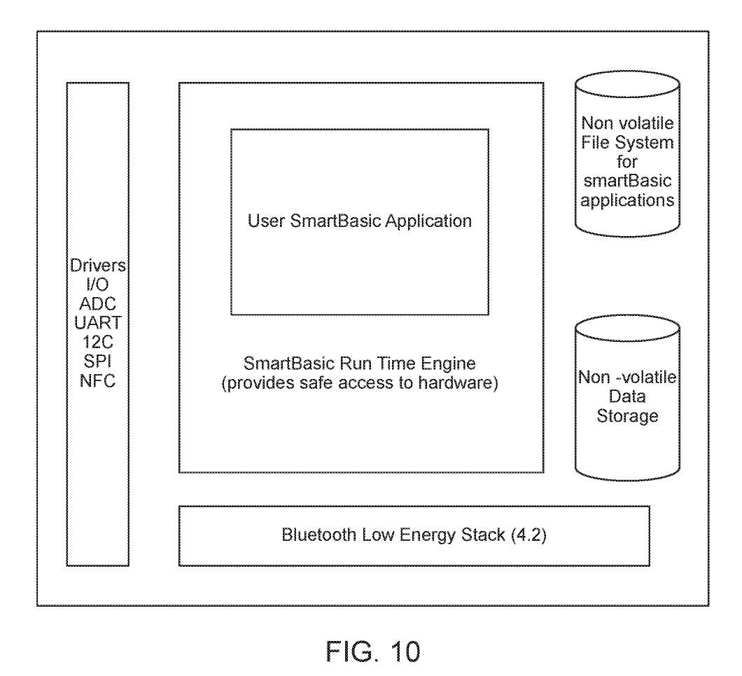

The communications module 815 can be a Bluetooth Low Energy (BLE) plus Near Field Communication (NFC) module with onboard antenna, such as a Laird BL652. The communication module 815 (e.g. Bluetooth Smart module) can include configurable interfaces providing UART, 12c, SPI, ADC, GPIO, PWM, FREQ, and NFC. FIG. 8A illustrates a top-down view and FIG. 8B is a bottom view of the BLE module 815 and FIG. 9 is a block diagram and pin out of the communication module 815. FIG. 10 is a block diagram of the functional hardware and software for the BLE module 815. The communication module 815 can include a chip antenna, antenna connector, and RF shield. Pin 1 can be a GND; Pin 7 can be nRESET; Pin 17 can be UART RX; Pin 18 can be UART_CTS; Pin 19 can be UART_TX; Pin 20 can be UART_RTS; Pin 23 can be SIO_2 (VSP_EN); Pin 26 can be VDD_nRF; and PIN 28 can be nAutorun. The communication module 815 can withstand an industrial temperature rating of −40° C. to 85° C. Despite the communication module 815 being rated only to 85° C., its configuration within the housing 700 allows for use up to at least 150° C.

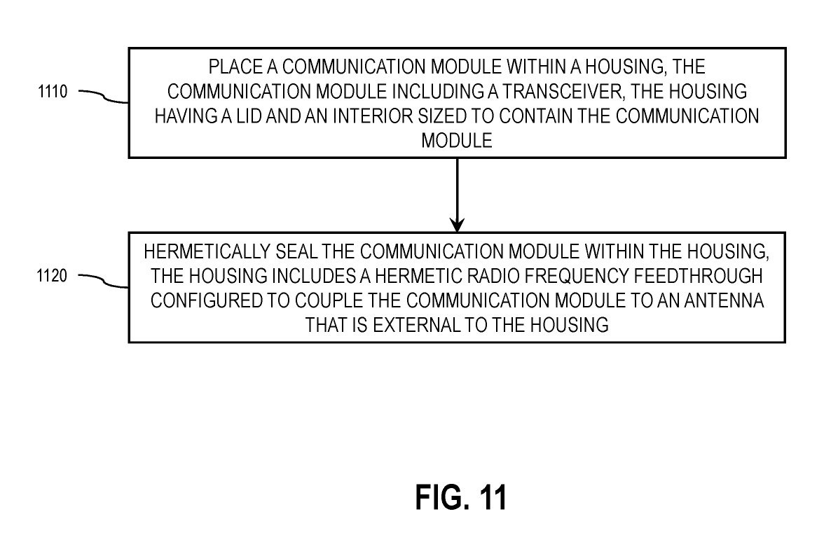

FIG. 11 is a flowchart illustrating a process 1100 for forming a sterilizable wireless communication device, in accordance with some example implementations. At operational block 1110 the process 1100 may include placing a communication module within a housing. The communication module may include a transceiver. The housing may have a lid and an interior sized to contain the communication module. At operational block 1120, the process 1100 may include hermetically sealing the communication module within the housing. The housing may further include a hermetic radio frequency feedthrough configured to couple the communication module to an antenna that is external to the housing.

Any of the instruments described herein can, but need not be coupled to robotic arms or robotic systems or other computer-assisted surgical systems in which the user uses a computer console to manipulate the controls of the instrument. The computer can translate the user's movements and actuation of the controls to be then carried out on the patient by the robotic arm. Robotics can provide real-time intra-operative tactile and/or auditory feedback along with visualization, such as three-dimensional modeling. The robotic system can have an articulated endowrist at the end of one or more “working” arms configured to be inserted through a small portal. A stable, camera arm with two lenses (allowing stereoscopic images) can be also inserted through another small portal. The end-effectors can manipulate instruments and can have various degrees of freedom. The user can control these endowrists individually or through a console placed in the operating room, allowing control of both the external and internal surgical environments. The user's interface can have instrument controllers that can filter tremor and decrease the scale of motion. Foot pedals can expand the user's repertoire, allowing tissue coagulation and irrigation. Visual feedback can be through a stereoscopic display. Robotic systems to which the devices disclosed herein can be coupled include the Haptic Guidance System or RIO® Systems (MAKO Surgical Corp, Ft. Lauderdale, Fla.), Navio Surgical System (Smith and Nephew), the da Vinci® Surgical Systems (Intuitive Surgical, Sunnyvale, Calif.), other surgical robots can be considered as well including the Robot-Assisted Micro-Surgery (RAMS) system (MicroDexterity Systems, Inc.), NeuroArm® (University of Calgary), Zeus® Surgical robots, SpineAssist (Mazor Surgical Technologies, Israel), ROBODOC and ORTHODOC (Curexo Technology Corp., Fremont, Calif.), ACROBOT (Acrobot, Elstree, UK), PathFinder (Prosurgics Ltd., Loudwater, High Wycombe, UK), and Laprotek system (Hansen Medical, Inc.). Other surgical instruments can be used with the sterilizable wireless communications devices described herein such that the instruments can be independently controlled by the surgeon in the field or at a robot console.

Aspects of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device. For example, a software program can be incorporated into the device that takes advantage of the reproducible relationship between energy, material strength and density. Energy is proportional to bone strength and density. As such the software can correlate the energy during drilling, driving, or sawing to the material strength and bone density. Such a software program can be used to measure material strength and bone density in real-time by determining the energy used by the working tool. The software program can also be used to control RPM, feed rate, current, voltage, and/or force on the working tool.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

Described herein are sterilizable wireless communication devices. In some implementations, the device includes a communication module having a transceiver capable of direct wireless communication and an antenna connected to the transceiver. The device includes one or more input/output connectors configured to directly communicate with an electronic device. The device also includes a housing having an interior sized to contain the communication module. The communication module is hermetically sealed within the housing.

The housing can include a window formed of a material that allows transmission and reception of radio waves from the antenna within the interior of the housing to outside of the housing. The housing can be a microelectronic hermetic housing with gas tight glass to metal sealing and ceramic to metal housing. The window can be a sapphire window fused to the hermetically sealed housing. The hermetically sealed housing can have a cobalt lid and the sapphire window can be fused to the cobalt lid. The housing can be a microelectronic hermetic housing with a full ceramic housing. The device can be sterilized by autoclave steam sterilization, ethylene oxide sterilization, chlorine dioxide sterilization, hydrogen peroxide sterilization, vaporized hydrogen peroxide sterilization, hydrogen peroxide plasma sterilization, gamma ray sterilization, and/or electron beam sterilization. The communication module can be a Bluetooth Low Energy module. The device can be incorporated inside a body of a medical instrument.

In an interrelated aspect, described is a sterilizable wireless communication device having a communication module capable of direct wireless communication, one or more input/output connectors configured to directly communicate with an electronic device, and a housing having an interior sized to contain the communication module. The communication module is hermetically sealed within the housing.

The communication module can include a transmitter, a receiver, or a transceiver. The communication module can be a transceiver and the device can include an antenna that is external to the hermetic seal and connects to the transceiver through an I/O. The communication module can include an antenna connected to a transceiver. The housing can include a window formed of a material that allows transmission and reception of radio waves from the antenna within the interior of the housing to outside of the housing. The housing can be a microelectronic hermetic housing with gas tight glass to metal sealing and ceramic to metal housing. The window can be a sapphire window fused to the hermetically sealed housing. The hermetically sealed housing can have a cobalt lid and the sapphire window can be fused to the cobalt lid. The housing can be a microelectronic hermetic housing with a full ceramic housing. The device can be sterilized by autoclave steam sterilization, ethylene oxide sterilization, chlorine dioxide sterilization, hydrogen peroxide sterilization, vaporized hydrogen peroxide sterilization, hydrogen peroxide plasma sterilization, gamma ray sterilization, and/or electron beam sterilization. The communication module can include a Bluetooth Low Energy module. The device can be incorporated inside a body of a medical instrument.

While this specification contains many specifics, these should not be construed as limitations on the scope of what is claimed or of what may be claimed, but rather as descriptions of features specific to particular implementations. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

What is claimed is:

1. A sterilizable wireless communication device, comprising:
a communication module comprising a transceiver capable of direct wireless communication; and
a housing having an interior sized to contain the communication module, wherein the communication module is hermetically sealed within the housing, and wherein the housing comprises a hermetic radio frequency feedthrough configured to couple to an antenna that is external to the housing.

2. The sterilizable wireless communication device of claim 1, wherein the housing is a microelectronic hermetic housing with a gas tight glass to metal sealing and a ceramic to metal housing.

3. The sterilizable wireless communication device of claim 1, wherein the communication module is coupled to the hermetic radio frequency feedthrough by a radio frequency cable.

4. The sterilizable wireless communication device of claim 3, wherein the radio frequency cable comprises a connector configured to mate with and couple to the hermetic radio frequency feedthrough.

5. The sterilizable wireless communication device of claim 4, wherein the hermetic radio frequency feedthrough comprises a sub-miniature push-on, micro (SMPM) connector.

6. The sterilizable wireless communication device of claim 1, wherein the housing comprises a stainless steel lid, wherein the stainless steel lid is laser welded to the housing.

7. The sterilizable wireless communication device of claim 1, wherein the housing is a microelectronic hermetic housing with a full ceramic housing.

8. The sterilizable wireless communication device of claim 1, wherein the housing is configured to be sterilized by autoclave steam sterilization, ethylene oxide sterilization, chlorine dioxide sterilization, hydrogen peroxide sterilization, vaporized hydrogen peroxide sterilization, hydrogen peroxide plasma sterilization, gamma ray sterilization, and/or electron beam sterilization.

9. The sterilizable wireless communication device of claim 1, wherein the communication module comprises a Bluetooth Low Energy module.

10. The sterilizable wireless communication device of claim 1, wherein the device is incorporated inside a body of a medical instrument.

11. A sterilizable wireless communication device, comprising:
a communication module capable of direct wireless communication;
one or more input/output connectors configured to directly communicate with an electronic device; and
a housing having an interior sized to contain the communication module, wherein the communication module is hermetically sealed within the housing.

12. The sterilizable wireless communication device of claim 11, wherein the communication module comprises a transceiver.

13. The sterilizable wireless communication device of claim 12, further comprising an antenna that is external to the hermetic seal and connects to the transceiver through an input/output connector of the one or more input/output connectors.

14. The sterilizable wireless communication device of claim 13, wherein the input/output connector comprises a hermetic radio frequency feedthrough.

15. The sterilizable wireless communication device of claim 11, wherein the communication module comprises an antenna connected to a transceiver.

16. The sterilizable wireless communication device of claim 15, wherein the housing comprises a window formed of a material that allows transmission and reception of radio waves from the antenna within the interior of the housing to outside of the housing.

17. The sterilizable wireless communication device of claim 16, wherein the housing is a microelectronic hermetic housing with gas tight glass to metal sealing and ceramic to metal housing.

18. The sterilizable wireless communication device of claim 16, wherein the window is a sapphire window fused to the hermetically sealed housing.

19. The sterilizable wireless communication device of claim 18, wherein the hermetically sealed housing has a lid and the sapphire window is fused to the lid.

20. The sterilizable wireless communication device of claim 19, wherein the lid comprises stainless steel and/or cobalt.

21. The sterilizable wireless communication device of claim 11, wherein the housing is configured to be sterilized by autoclave steam sterilization, ethylene oxide sterilization, chlorine dioxide sterilization, hydrogen peroxide sterilization, vaporized hydrogen peroxide sterilization, hydrogen peroxide plasma sterilization, gamma ray sterilization, and/or electron beam sterilization.

22. The sterilizable wireless communication device of claim 11, wherein the communication module comprises a Bluetooth Low Energy module.

23. The sterilizable wireless communication device of claim 15, wherein the device is incorporated inside a body of a medical instrument.

24. A method for forming a sterilizable housing for a wireless communication device, the method comprising:
placing a communication module within a housing, the communication module comprising a transceiver, the housing having a lid and an interior sized to contain the communication module; and
hermetically sealing the communication module within the housing, the housing further comprising a hermetic radio frequency feedthrough configured to couple the communication module to an antenna that is external to the housing.
